(19) United States
(12) Patent Application Publication
(10) Pub. No.: US 2003/0113312 A1
(43) Pub. Date: Jun. 19, 2003

(54) Skin barrier function and cohesion through enhanced stratum corneum acidification

(75) Inventors: Peter M. Elias, Mill Valley, CA (US); Kenneth R. Feingold, San Rafael, CA (US); Joachim W. Fluhr, San Francisco, CA (US); Theodora M. Mauro, San Francisco, CA (US); Martin J. Behne, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA

(21) Appl. No.: 10/017,038

(22) Filed: Dec. 14, 2001

(51) Int. Cl. A61K 38/46; A61K 31/685; A61K 31/66

(52) U.S. Cl. 424/94.6; 514/114; 514/78

(57) ABSTRACT

The integrity, barrier function, cohesion and antimicrobial defense of the stratum corneum pH are improved by acidification, which is achieved by the application of low pH buffers, enzymes, phospholipids or salts with monovalent cations.

### STATEMENT AS TO RIGHTS TO INVENTIONS MADE UNDER FEDERALLY SPONSORED RESEARCH AND DEVELOPMENT

This invention was made at least in part with assistance from the United States Federal Government, under Grant No. AR 19098 from the National Institutes of Health. As a result, the government may have certain rights to this invention.

This invention resides in the technical field of topical formulations for application to skin or mucous membranes, and to the treatment of subjects suffering from skin or mucous membrane diseases or disorders of epidermal barrier function and cohesion and of subjects suffering from conditions that display abnormalities of barrier function.

### BACKGROUND OF THE INVENTION

Mammalian epidermis consists of a continuously self-replicating, stratified, keratinized squamous epithelium, the principal cells of which are keratinocytes. The population of keratinocytes undergoes continuous renewal throughout life. A mitotic layer of basal cells replaces cells at the surface as they slough off. As they move above the basal layer of the epidermis, keratinocytes undergo a process of differentiation known as keratinization. They undergo progressive changes in shape and content, eventually transforming from polygonal living cells, into anucleate, non-viable, flattened squames replete with keratin and other proteins. The stratum corneum is the outermost layer of the epidermis and the final product of epidermal differentiation.

The mammalian epidermis serves many functions, amongst which are formation and maintenance of a cohesive permeability barrier that guards against excessive transcutaneous water loss and as an external barrier against microbial attack. The stratum corneum of mammalian skin displays a strongly acidic pH. The pH of the upper stratum corneum measures approximately 4.5-5.0 while the pH of the lower stratum corneum (above the outermost granular cell layer) approaches neutrality. Thus, the stratum corneum experiences a pH differential of more that two pH units over a vertical space of less than 100 microns; a dramatic biological phenomenon. This pH gradient occurs not only in human, but also in rodent skin despite its much thinner stratum corneum.

Although first recognized decades ago (Shade, H., et al., Klin. Wochenschr. 7:12-14 (1928)), understanding of the origin and function of the “acid mantle” of the stratum corneum is still incomplete. Although acidity is thought to be essential for certain stratum corneum functions, the “acid mantle” is not seen as being generated by the cells of the stratum corneum itself. Some investigators have proposed mechanisms for the origin and maintenance of an acidic stratum corneum that combine exogenous and endogenous processes. Ohman, H., et al., J. Invest. Dermatol. 111:674-7 (1998) propose that the acid mantle of the stratum corneum is a combined effect of acidic excretion products from sweat and sebum and the hydrolytic products of filaggrin breakdown (urocanic and pyrrolidone carboxylic acid) originating in the granular layer and further concentrated in the upper stratum corneum as a result of desiccation.

The inventors have shown that at least two other mechanisms contribute to formation and maintenance the acid mantle of the stratum corneum. Mauro, T., et al., Arch. Dermatol. Res. 290:215-222 (1998) who showed that maintenance of extracellular pH regulates barrier homeostasis by controlling the post-secretory processing of lipid precursors, which are degraded by enzymes with an acidic pH optimum. In their model, lipids secreted at the stratum corneum-stratum granulosum interface are first processed by enzymes with a neutral pH optima. As the lipids processed by these enzymes migrate further outward, the extracellular environment becomes more acidic and this in turn activates enzymes such as β-glucocerebrosidase and perhaps other enzymes with acidic pH optima. Thus, the acidity of the stratum corneum is essential for at least one function; maintenance of barrier homeostasis.

Whatever the nature of its origin, it is known that the acid mantle is essential to normal functioning of the stratum corneum. Several pathological situations reveal the importance of the acidic stratum corneum. An acidic pH inhibits colonization by pathogenic bacteria such as S. aureus and alkalization of the stratum corneum, as occurs in urea-soaked skin of diaper dermatitis, is an important antecedent of bacterial and yeast infections. In the elderly, where the epidermal pH is more alkaline, the stratum corneum displays a lowered buffering capacity and barrier repair is perturbed making skin more susceptible to disease. Acidification regulates cohesion of the stratum corneum as well as barrier function. The activities of several proteases that are important for desmosomal cleavage are modulated by pH, functioning optimally at neutral pH. Hence, altered acidification is associated with abnormal stratum corneum cohesion in disorders such as X-linked ichthyosis. Acidification thus regulates desquamation by inhibiting the activities of these pH sensitive proteases, preventing premature desmosomal cleavage and degradation.

The acid mantle of the stratum corneum is thus critical for the maintenance of the cutaneous permeability barrier, for optimal stratum corneum cohesion and for cutaneous antimicrobial defense. However, the origin and function of the acid mantle of the stratum corneum is still incompletely understood.

### SUMMARY OF THE INVENTION

It has now been discovered that inhibition or deletion of the NHE1 sodium/hydrogen antiporter is associated with an increase of the pH within the stratum corneum and that this pH increase leads to impaired stratum corneum barrier function and reduced stratum corneum cohesion. In accordance with this invention, therefore, the NHE1 antiporter is regulated to increase stratum corneum acidification by the application of exogenous NH₄⁺ ion, or by the application of exogenous Na⁺ ion.

It has also been discovered that applications of secretory phospholipase inhibitors increase stratum corneum alkalinity by blocking the generation of free fatty acids, giving rise to abnormalities in barrier homeostasis and cohesion. Accordingly, this invention further resides in the topical administration of secretory phospholipase A₂, phopholipids or a combination of secretory phospholipase A₂ and its phospholipid substrates, to achieve acidification and improve barrier function, cohesion, and integrity of the stratum corneum.

The invention also resides in the application of buffers with a pH less than 7.0 to achieve acidification of the stratum corneum.

The invention therefore resides in the topical application of substances that acidify the stratum corneum with the purpose of enhancing the permeability barrier and improving cohesion, integrity and antimicrobial defense. Some of the applied substances are salts with monovalent cations, notably Na⁺ and NH₄⁺ salts, which dissociate into topical monovalent cations and which in turn stimulate proton generation via the NHE1 sodium/proton antiporter. Other substances of this invention are buffers with a pH of less than 7.0, phospholipids and phospholipases, either alone or in combination with phospholipid substrates.

Other features, embodiments and advantages of the invention will become apparent from the description that follows.

### BRIEF DESCRIPTION OF THE DRAWINGS

FIG. 1 is a plot of pH vs. time measured as the fluorescence at 530 nm from cells labeled with the pH sensitive dye BCECF. Activity of the NHE antiporter was tested by measuring the cell's ability to recover from an acid load. The plot shows pH (x-axis) vs. time (y-axis, seconds). (A) starting equilibration. Arrow indicates the addition of NH₄Cl, followed by a spike of alkalinization and after dissociation of the NH₄⁺ ion, an acid load inside the cells. Cells slowly extrude H⁺ reestablishing a more neutral pH (B). The addition of amiloride at 1 μM (C) blocks the recovery from an identical acid load. (D) The wash out period shows recovery from the acid load indicating a non-permanent, non-toxic effect of amiloride.

FIG. 2 is a bar graph showing the changes in pH of the stratum corneum after skin is tape stripped then exposed to acidic (pH 5.5) or neutral (pH 7.4) buffers with or without NHE 1 inhibitors. For each condition tested, e.g. pH 5.5, the graph depicts the surface pH at 2 hours, 5 hours, and 24 hours post-tape-strip.

### DETAILED DESCRIPTION OF THE INVENTION AND SPECIFIC EMBODIMENTS

The acidic pH environment of the stratum corneum is important for epidermal biology. Functions such as stratum corneum cohesion and desquamation, the formation of an epidermal permeability barrier and formation of the antimicrobial barrier all depend on the pH gradient.

It has now been discovered that through its action in the hydrolysis of phospholipids, phospholipase is critical for the formation and maintenance of the acidic pH gradient of the stratum corneum. Phospholipase contributes to the generation and maintenance of the acid pH environment of the epidermis through the production of fatty acids which result from the hydrolysis of its phospholipid substrates. According to one aspect of this invention, therefore, a phospholipase, the phospholipid substrate of a phospholipase, or the enzyme and the phospholipid in combination, are applied to the epidermis in amounts that are effective at improving the permeability and antimicrobial barrier of the stratum corneum. Preferred phospholipases are phospholipase A₁ and phospholipase A₂. Preferred phospholipase A₂'s are natural or bioengineered 14 kDA type 1 secretory phospholipase A₂ and secretory phospholipase A₂ pancreatic type. Acidification that results from phospholipase activity also improves cohesion and integrity of the stratum corneum.

The acid pH environment of the epidermis is also improved and maintained in accordance with this invention by the administration of phospholipids, as substrates for phospholipases that are already present in the epidermis. Preferred phospholipids are non-essential fatty acid-containing phospholipids, and a particularly preferred phosphpolipid is dipalmitoylphosphatidylcholine.

It has also been discovered that the sodium/hydrogen antiporter, NHE1, is located in the outer differentiated layers of the epidermis and that its activity contributes significantly to acidification of the stratum corneum. According to one aspect of this invention, activity of the sodium/hydrogen antiporter, NHE1, is stimulated through the application to the epidermis of salt with monovalent cations, preferably sodium salts or ammonium salts, in amounts that are effective at improving the permeability and antimicrobial barrier. These salts include both organic and inorganic salts, and are administered in accordance with the invention in amounts that are effective for improving cohesion and integrity of the stratum corneum. Preferred salts are those in which the counter ion is other than lactate ion. Examples of inorganic salts that can be used in the practice of this invention are ammonium chloride, ammonium phosphate, ammonium carbonate, ammonium nitrate, ammonium sulfate, ammonium sulfanate, ammonium fluoride, ammonium iodide and ammonium bromide sodium chloride, sodium phosphate, sodium carbonate, sodium nitrate, sodium sulfate, sodium sulfanate, sodium fluoride, sodium iodide and sodium bromide. A particularly preferred inorganic salt is ammonium chloride. Examples of organic salts that can be used in the practice of this invention are ammonium citrate, ammonium tartrate, ammonium acetate, sodium citrate, sodium tartrate, and sodium acetate.

In another aspect of this invention, buffers with a pH of less than 7.0 are applied to the epidermis to improve the permeability and antimicrobial barrier. In another aspect, buffers with a pH of less than 7.0 are applied to the epidermis in amounts that are effective for improving cohesion and integrity of the stratum corneum. Examples of such buffers are HEPES-based buffers (where HEPES is N-[2-hydroxyethyl]piperazine-N′-[2-ethanesulfonic acid]), MES-based buffers (where MES is 2-(N-morpholino)ethanesulfonic acid), MOPS-based buffers (where MOPS is 3-(N-morpholino)propanesulfonic acid), PIPES-based buffers (where PIPES is 1,4-piperazinediethanesulfonic acid), TES-based buffers (where TES is N-tris[hydroxymethyl]methyl-2-aminoethanesulfonic acid), phosphate-based buffers, citrate-based buffers or bicarbonate-based buffers. HEPES-based buffers are particularly preferred. The pH of the buffer may vary, but best results will generally be achieved with a buffer pH in the range of 4.5-6.5. A particularly preferred buffer pH is 5.5.

This invention also extends to topical compositions that contain two or more active ingredients from among the various types and examples set forth above.

This invention is useful for the treatment of diseases that involve perturbed epidermal barrier function, reduced stratum corneum cohesion and integrity and inadequate antimicrobial defense. Treatment can be achieved for example by administration of:

phospholipids alone,

phospholipids in conjunction with secretory phospholipase A₂,

secretory phospholipase A₂ alone,

salts with monovalent cations, or

acidic pH buffers

Examples of conditions that are treatable by this invention are psoriasis, premature infant's skin, atopic dermatitis, keloids, hypertrophic scars, metabolic lipidosis, burn wounds, occupational dermatitis, allergic contact dermatitis and irritant contact dermatitis.

In other embodiments, this invention resides in the administration of the agents listed above for the treatment of skin conditions that involve abnormal stratum corneum cohesion. Examples of such conditions are various types of ichthyosis with or without an associated barrier abnormality, aged skin, xerosis, benign neoplasms such as warts, and seborrheic keratoses.

In still other embodiments, this invention resides in the administration of the agents listed above for the treatment and prevention of skin conditions that involve weakened antimicrobial defense. Examples of such conditions are the skin of diabetic patients with increased susceptibility to microbial infections, diaper dermatitis, eczema, (which becomes more inflammatory at neutral pH), candidiasis, healing wounds and in immune-compromised individuals suffering from HIV disease or post-transplant immunosuppression therapy.

In the practice of this invention, the acidifying agents will be administered as active ingredients in a formulation that is pharmaceutically acceptable for topical administration. These formulations may or may not contain a vehicle, although the use of a vehicle is preferred. Preferred vehicles are non-lipid vehicles, particularly a water-miscible liquid or mixture of liquids. Examples are methanol, ethanol, isopropanol, ethylene glycol, propylene glycol, and butylene glycol, and mixtures of two or more of these compounds.

The concentration of active ingredient in the vehicle is not critical to this invention and may vary widely while still achieving a therapeutic effect, a preventive effect, or both. In most cases, the concentration will fall within the range of from about 10 μM to about 1,000 μM, although for certain active ingredients, the concentration may vary outside this range. For example, the preferred concentrations of some active ingredients will be within the range of about 10 μM to about 1,000 μM, and in others the preferred range will be 100 μM to about 1,000 μM.

The invention is generally applicable to the treatment of the skin of terrestrial mammals, including for example humans, domestic pets, and livestock. The invention is of particular interest in treating humans for the conditions described above or for preventing these conditions from becoming manifest.

Materials and Methods for Examples 1 through 10

Animals: Male hairless mice (Skh1/Hr, Charles River Laboratories, Wilmington, Mass.), 8-12 weeks old, were fed Purina mouse diet and water ad libitum. Heterozygous, NHE1 deficient mice (Bell et. al., 1999) were bred in house, from founders received from Dr. G. E. Shull, Cincinnati, Ohio. Each litter was genotyped separately. Functional experiments were performed on animals aged 6-10 weeks.

Chemicals: Propylene glycol, ethanol, NaOH and HCL were from Fischer Scientific. Amiloride (N-Amido-3,5-diamino-6-chloropyrazinecarboxamide), bromphenacyl bromide (BPB), HEPES (N-[-2-Hydroxyethyl]piperazine-N′-[2-ethanesulfonic acid]) and TES (N-tris[Hydroxymethyl]methyl-2-aminoethanesulfonic acid) were purchased from Sigma Chemical Co. Bio-Rad Protein Reagent, Bradford protein assay kits, lyophilized bovine plasma gammaglobulin (GG) and Bovine Serum Albumin were purchased from Bio-Rad Laboratories. 2′,7′-bis(carboxyethyl)-5,6-carboxyflourescein-AM ester (BCECF-AM) was from Molecular Probes (Eugene, Oreg.). HOE694 (3-methylsulfonyl-4-piperidino-benzoylguanidinemethanesulfonate) was kindly provided by Dr. H. J. Lang, Hoescht-Marion-Roussel AG, Frankfurt, Germany. One-hexadecyl-3-triflouroethylglycero-sn-2-phosphomethanol (MJ33) was synthesized as described previously (Jain, M. K., et al., Biochemistry 30:10256-68 (1991)). The stripping for the protein assay was performed with 22 mm D-Squame 100 tapes purchased from Cu-Derm (Dallas, Tex.).

Permeability Barrier Studies: Normal hairless mice were treated topically twice a daily for three days with BPB (4 mg/mL) or MJ33 (4 mg/mL) both in propylene glycol:ethanol (7:3 v/v) vehicle, or the vehicle alone on an area of 5-6 cm on the backs and flanks as described previously (Mao-Qiang, M., et al., J. Lipid Res. 36:1926-35 (1995)). The doses employed were shown previously to be non-toxic to Murine skin (Mao-Qiang, M., et al. (1995); Mao-Qiang, M., et al., J. Invest. Dermatol. 106:57-63 (1996)), and to inhibit secretory phospholipase A₂ (sPLA₂) activity selectively in different cell types (Gelb, M. H., et al., Faseb J. 8:916-24 (1994); Jain, M. K., et al. (1991)). The acidification experiments were performed with 10 mM HEPES-buffer, adjusted to either pH 5.5 or pH 7.4 as follows: One flank of anesthetized mice was immersed on a mesh netting, as described previously (Lee, S. H., et al., J. Clin. Invest. 89:530-8 (1992)). The mice were anesthetized with chloral hydrate (Morton Grove Pharmaceuticals, Morton Grove, Ill., USA). After 3 hours immersion at 37° C., the mice were removed, and the remaining buffer was gently blotted off. After 15 minutes, barrier function was determined by measurement of transepidermal water loss (TEWL) with an electrolytic water analyzer (MEECO®, Warrington, Pa.). Surface pH was measured with a flat, glass surface electrode from Mettler-Toledo (Giessen, Germany), attached to a pH meter (Skin pH Meter PH 900, Courage & Khazaka, Cologne, Germany). Individually tested sites were covered with Hilltop Chambers (HtC, nominal volume 200 mL), which were reapplied following each indvidual measurement. For topical applications, solutions of HEPES buffer (10 mM) at either pH 7.4 or pH 5.5, contained either Amiloride (5 mM), HOE694 (7.5 mM), or buffer alone. The inhibitor-concentrations represented the published 50% inhibitory concentrations (IC₅₀, values in mM) for the NHE 1 inhibitory compounds in fibroblasts.

The stratum corneum was removed by several strippings with adhesive tape (Tesa, Beiersdorf, Germany) inducing an increase in TEWL rates above baseline (from 0.2 to approximately 7-9 g/m²/h). Two sites were prepared on each animal, and TEWL and surface pH were measured at 0, 2, 5 and 24 hrs following the stripping and the applications of inhibitors. Biopsies were taken for electron microscopy from treated and control sites, at 5 and 24 hours. For studies in the NHE1 knockout mice, homozygous (−/−) mice were compared to their wildtype (+/+) littermates. Flanks of these mice were shaved, and barrier homostasis was studied 48 hours later. For tape-stripping of these animals, D-Squame disks (Cu-derm, Dallas, Tex.) were used, as Tesa tape was too disruptive for application to shaved, hairy mouse skin.

Cell Culture and Western Immunoblotting: Second passage cultured human Keratinocytes from human foreskin (CHK) were grown in low calcium medium (0.03 mM Ca²⁺, Cascade 154, Cascade Biologics, Eugene, Oreg.) until they reached approximately 60% confluence. Cells were then incubated with various concentrations of HOE694, and compared to cell grown in high calcium medium (1 mM or 2 mM Cascade 154) with HOE694. Following incubations of 48 hours, CHK were harvested and frozen in liquid nitrogen

The cells were thawed, homogenized by sonication. Their protein content determined, and gels were loaded with equal amounts per sample and lane. Western Immunoblotting was performed using 7.5% SDS-PAGE, as described previously (Laemmli, 1970). Following transfer of protein to PVDF membranes, blots were incubated overnight with primary antibody at 4° C. (monoclonal anti-INV, clone SY5, Sigma Immunochemicals, St Louis, Mo.). Secondary antibody was applied and blots were incubated at room temperature for 2 hours (peroxidase conjugated anti mouse; Amersham PharmaciaBiotech Inc., Piscataway, N.J.). Final detection was performed by chemiluminescence (ECL kit; Amersham). NHE1 was detected in membrane fractions prepared from CHK (cultured as follows: undifferentiated keratinocytes were cultured with 0.03 mM calcium until reaching ˜80% confluence; differentiated cells were maintained in 1.2 mM until reaching either 4 or 7 days post confluence). The primary antibody used was mouse monoclonal anti-NHE1 (Chemicon Int., Temecula, Calif.). For AB/AG competition studies, the primary antibody was preabsorbed with the peptide used for creating the antibody (Alpha Diagnostic, San Antonio, Tex.).

Ultrastructural methods: Freshly obtained biopsies from mouse skin were fixed directly in modified Karnovsky's fixative, postfixed with reduced osmium tetroxide (OS0₄) and then imbedded in an Epon-epoxy mixture. For visualization of lipid-enriched, lamellar bilayer structures, some samples were post fixed in ruthenium tetroxide (RuO₄). Sections were cut on a Reichert Ultracut E microtome, counterstained with uranyl acetate and lead citrate, and viewed in a Zeiss 10 CR electron microscope, operated at 60 kV.

Immunohistochemistry: Fresh samples of normal human skin from surgical margins, or biopsies from NHE1 +/+ or −/− mice were formaldehyde fixed, parafin embedded, and sectioned (5 μm). For immunolabeling of NHE1, a rabbit polyclonal antibody was used (Chemicon Int., Temecula, Calif.), which was detected via FITC-labled, secondary goat anti-rabbit antibody (Cappel, Organanon Teknika Corp., Durham, N.C.). Sections were counterstained with propidium iodide (Sigma, St. Louis, Mo.), and pictures were taken on a Leica TCS-SP confocal microscope.

Immunofluorescence Staining: Hairless mouse skin was excised with a 6 mm punch biopsy, and the subcutaneous fat was removed. Tissue sections were incubated for 1 hour in blocking buffer (1% bovine serum albumin, 0.1% cold water fish gelatin in phosphate buffered saline (PBS)), and were then incubated for 1 hour further at room temperature with 1:500 dilution of polyclonal rabbit anti-mouse desmoglein 1 antibody (gift of Dr. John Stanley, University of Pennsylvania) diluted in blocking buffer. The tissue was then washed with blocking buffer and incubated one hour at room temperature with floroscien-labeled, isothiocyanate-conjugated, goat anti-rabbit IgG antibody (DAKO, Carpinteria, Calif., USA), diluted in blocking buffer. Either preincubation of DSG 1 antibody with DSG 1-recombinant protein (gift of Dr. Masayuki Amagai, Keio University, Tokyo, Japan), or omission of the DSG 1 primary antibody eliminated specific staining. Tissue sections then were washed with PBS and coverslipped before visualization under confocal microscope (Leica TCS SP, Heidlberg, Germany) using FITC at an excitation wavelength of 494 nm and an emisson wavelength of 518 nm.

Protein Assay on Sequential Tape Strips: The protein assay utilized the Bradford dye-binding procedure for quantification of total protein (Bradford, 1976). HEPES buffer and propylene glycol:ethanol, the two vehicles used in these studies, are known to be compatible with this assay. Before stripping the stratum corneum, the skin surface was cleaned with a single ethanol wipe. D-Squame tapes then were placed sequentially to the test areas for about 3 sec each, removed with forceps, and stored in glass scintillation vials at 5° C.

The amount of protein removed per D-Squame was measured, by a modification of the method of Dreher, F., et al., Acta Derm. Venereol 78:186-9 (1998). The microassay system was shown to be linear in the range of 1-10 μg/mL, using human stratum corneum removed from a heel callosity. The protein content per stripping was determined with the Bio-Rad protein assay kit. Lyophilized, bovine gammaglobulin (GG) was used as standard in all assays, because it correlated best with human stratum corneum. Each tape was incubated with 1 M NaOH for 1 hour at 37° C. in an incubator shaker at 80 rpm, and neutralized thereafter with 1 mL of 1 M HCl in the scintillation vials. Subsequently, 0.2 mL of this solution was incubated in 0.6 ml distilled water plus 0.2 mL of the Bio-Rad protein dye for 5 minutes in boroscillate tubes. After incubations, the reagents were transferred to polystyrene cuvettes, and absorption was measured with a Genesys 5 spectrophotometer (Spectronic, Rochester, N.Y.) at 595 nm. An empty D-Squame tape, as well as distilled water incubated with Bio-Rad dye, served as negative controls. The amount of calculated protein was then normalized to skin surface area (μg/cm²). The amount of removed protein per D-Squame strip agreed with previous reports in untreated skin of hairless mice (i.e., range of 2.5-4 μg/strip) (Weber, S. U., et al., J. Invest. Dermatol. 113:1128-32 (1999)).

Assessment of Intracellular pH: Keratinocytes were plated on glass coverslips and grown in Cascade 154 medium containing 0.07 mM Ca²⁺. The coverslips were incubated in 145 μM BCECF-AM ester at room temperature for 5 minutes, then rinsed for 30 minutes in buffer containing either 28 mM HEPES, 136 mM NaCl, 5 mM KCl, 0.07 mM CaCl₂ and 10 mM glucose, pH 7.4 or Ringer solution (136 mM NaCl, 5 mM KCl, 0.03 mM Ca2+, 28 mM TES, 14 mM Na HCO₃, NaOH to adjust the pH to 7.4, and 10 mM glucose (pH 7.4, 330 mOsm)). BCECF-AM-ester is membrane permeable; once inside cells it is de-esterified by endogenous carbonic anhydrase, trapping flourescently-active, acidic BCECF inside the cells. The BCECF-loaded cells were placed in a superfusion chamber mounted on a inverted microscope (Nikon). The keratinocytes were superfused with one of the two solutions, and baseline intracellular pH was monitored constantly using a ratiometric method (Paradiio et al (1987) Am J. Physiol. 253:C30-6). Briefly, BCECF loaded cells were alternately illuminated every 10 seconds with brief flashes (200 milliseconds) of 440 nm and 490 nm light. The resultant fluorescence (at 530 nm) from each stimulating wavelength was measured with a CCD camera (Hamamatsu) digitally ratioed, and stored using a software program (Fluor, Universal Imaging Co., West Chester, Pa.). Whereas fluorescence emission at 440 nm excitation remains constant, indicative of dye concentration, emission from 490 nm varies proportionally with changes intracellular pH. By ratioing the two signals, intracellular pH is measured as a brightness signal corrected for the intracellular dye concentration.

After equilibration under basal conditions, cells were either alkalinized or acidified by superfusion for two minutes with Ringer solution in which 20 mM NH₄Cl had been substituted for NaCl (i.e. 116 mM NaCl, 20 mM NH₄Cl), amiloride (μM) then was added to the bath solution. Calibration was performed at the end of each experiment by permeablizing the cells to protons with nigericin, thereby equilibrating intra- and extracellular pH. Superfusing the cells with solutions containing 85 mM NaCl, 50 mM KCl, 0.07 mM CaCl₂, 10 mM nigericin (a K⁺/H⁺ exchanger), and either 28 mM HEPES, or 28 mM TES bubbled with 5% CO₂, pH 6.8 or 7.8, allowed to calibrate intracellular signals.

mRNA Measurements by RT-PCR: Total RNA was prepared using the Qiagen RNeasy method (Qiagen, Valencia, Calif.) from both second passage CHK (grown in 1.2 mM Ca²⁺ to 4 days post confluence) or from human skin (from normal surgical margins) incubated with dispase (50 U/mL, Gibco, Life Technologies, Rockville, Md.) to prepare whole epidermis. RNA from each sample was reverse transcribed using Gibco reverse transcriptase. The cDNA was then amplified by PCR, employing the following primer set for human NHE1

5′ ACC CTG CTC TTC TGC CTC ATC G3′

5′ CCT GCT TCA TCT CCA TCT TGT G3′

The PCR product was separated on an agarose gel, purified, and subcloned into one shot competent cells (InVitrogen, Carlsbad, Calif.), plasmid DNA was prepared and sequenced. As a negative control, the same amplification without prior reverse transcription, gave no transcription product.

Statistical Analysis: Statistical analyses were performed using Prism 2 (GraphPad Software Inc., San Diego, Calif.). Normal distribution was tested before calculating the comparison. In the three group comparisons an ANOVA was first calculated followed by a post-hoc test (Bonferroni). Two groups were compared with an unpaired t-test.

### EXAMPLE 1

The experiments reported in this example demonstrate that inhibition of secretory phospholipase A₂ results in an increase in stratum corneum pH and that this pH increase is associated with impaired barrier function, reduced integrity and reduced cohesion of the stratum corneum.

Male hairless mice 8-12 weeks old were treated twice daily for three days with topical applications of the secretory phospholipase A₂ inhibitor bromphenacyl bromide (BPB) at a concentration of 4 mg/mL in propylene glycol:ethanol vehicle (7:3 v/v), or the vehicle alone, on an area of 5-6 cm on their backs and flanks, as described in materials and methods. After one day of treatment, the pH of the skin of the treated hairless mice increased significantly from a starting value of about pH 5.6 to nearly pH 6.0. Over the three day trial period, the pH of the BPB treated skin sites continued to show an increase relative to that of vehicle treated sites; the final average pH for treated sites on day 3 of the trial was nearly pH 6.4, whereas that of vehicle treated sites was close to pH 5.4.

By day 2 of the experiment, daily topical applications of secretory phospholipase A₂ inhibitor produced an abnormality in barrier function of the treated skin as measured by rates of transepidermal water loss (TEWL). TEWL rates for the treated sites were near 6 g/m²/h, whereas the vehicle treated sites showed TEWL levels closer to 2.5 g/m²/h. By day three of the experiment, TEWL rates increased to nearly 11 g/m²/h. In contrast, the vehicle treated sites had TEWL rates near 3 g/m²/h.

The integrity of the stratum corneum, evaluated as the number of tape strippings required to produce elevated TEWL rates, was markedly abnormal after three days of BPB treatment. A significant abnormality was present by the second stripping, and integrity continued to decline thereafter. By the second strip, sites treated with BPB had TEWL rates close to 20 g/m²/h, whereas vehicle treated sites had rates closer to 5 g/m²/h. This abnormality persisted and was amplified over the course of the experiment. By the fifth and final strip, TEWL rates were near 90 g/m²/h for the BPB treated sites and close to 25 g/m²/h for the vehicle treated sites.

The decline in integrity was paralleled by progressive loss of stratum corneum cohesion, as measured by quantification of the cumulative protein removed per D-Squame stripping. As was the case for pH increase, barrier function (measured as TEWL) and stratum corneum integrity, cohesion of the stratum corneum was impaired by the application of secretory phospholipase A₂ inhibitors. The amount of protein removed per D-Squame stripping was significantly greater from the experimental sites than the vehicle treated sites, even by the first strip; vehicle treated sites showed protein losses of about 5 μg/cm², whereas the losses from BPB treated sites were close to 20 μg/cm². This trend continued with further stripping. After five strips the vehicle treated sites lost about 20 μg protein/cm² of surface stripped, where the BPB treated sites lost about 85-90 μg protein/cm². Thus, applications of the secretory phospholipase A₂ inhibitor, BPB, result in an increased skin surface pH, and this pH increase is accompanied by altered barrier function, and also by reduced stratum corneum integrity and cohesion.

Because BPB is an alkylating agent, and could non-specifically affect other cellular processes, additional studies were performed using a chemically unrelated competitive inhibitor of secretory phospholipase A₂, MJ33. MJ33 is a highly specific inhibitor of group 1 secretory phospholipase A₂. Like BPB, MJ33 produced an increase in stratum corneum surface pH vs vehicle alone (5.87+/−0.06 vs 5.60+/−0.05; p=0.0023). Moreover, repeated applications of MJ33 produced a progressive abnormality in stratum corneum barrier function resulting in a 2-3 fold increase in TEWL rates by day three (5.19+/−0.81 vs. 2.97+/−0.16; p<0.001, for MJ33 vs. vehicle treated animals).

As with BPB treatments, three days of MJ33 applications progressively reduced stratum corneum integrity and cohesion. By day 3 of the experiment, TEWL rates for the MJ33 treated sites approached 75 g/m²/hr after three consecutive tape strips, while the vehicle treated sites showed TEWL rates closer to 40 g/m²/h. The reduced integrity of MJ33 treated sites was reflected by a parallel change in stratum corneum cohesion. By day 3 of the experiment, vehicle treated sites lost an average of about 20 μg protein/cm² after three consecutive tape strippings. In contrast, by day three of the experiment the MJ33 treated sites lost protein over 50 μg protein/cm² after three consecutive tape strippings.

These results show that two, chemically-unrelated secretory phospholipase A₂ inhibitors produce increases in skin surface pH, which are coupled to altered stratum corneum barrier function, integrity and cohesion.

### EXAMPLE 2

The experiments reported in this example show that the secretory phospholipase A₂ inhibitor induced decline in stratum corneum integrity and cohesion can be attributed to premature dissolution of desmosomes in the lower stratum corneum.

To further investigate the changes in stratum corneum cohesion that are brought about by application of secretory phospholipase A₂ inhibitors, BPB and MJ33 treated samples of murine skin and the corresponding untreated controls, were assessed by immunohistochemical staining and confocal microscopy. Both BPB and MJ33 provoked a dramatic reduction in the density of desmosomes in the lower stratum corneum and at the stratum corneum-stratum granulosum interface. In inhibitor treated sites, desmosomes were reduced in size and remnants of desmosomes were present at the stratum corneum-stratum granulosum interface. The number and appearance of desmosomes was normal in vehicle treated sites.

A reduction in desmosomes was also seen by immunohistochemical assessment of desmoglein-1 (DSG1) positive structures in the lower stratum corneum of BPB vs. vehicle treated stratum corneum. On laser confocal microscopy, the density of DSG1 positive clusters, which are presumed to correspond to intact desmosomes, decline dramatically in BPB treated stratum corneum, while DSG1 positive staining in vehicle-treated stratum corneum is comparable to control. These results show that the inhibitor induced decline in stratum corneum integrity and cohesion can be attributed to a premature dissolution of desmosomes in the lower stratum corneum.

### EXAMPLE 3

The experiments reported in this example show that exposure of murine skin to neutral pH buffer alone induces functional alterations in the stratum corneum that mimic changes induced by secretory phospholipase A₂ inhibitors.

Stratum corneum integrity and cohesion were examined after short term exposure of normal skin to neutral (pH 7.4) vs. acidic (pH 5.5) pH. After 3 hours of exposure to a neutral pH buffer (HEPES, pH 7.4), the surface pH of the stratum corneum rose from 5.86+/−0.21 to 6.41+/−0.20. Exposure to an acidic buffer lowered the surface pH from 5.95+/−0.05 to 5.72+/−0.06. Whereas exposure to both buffers increased transepidermal water loss (TEWL) rates, a slightly greater increase in TEWL occurred following three hour exposure to the neutral pH buffer (all changes normal range).

Stratum corneum integrity was also impaired by exposure to neutral (pH 7.4) buffer. TEWL was measured after epidermal sites which had been exposed to either neutral or acidic buffers, as described in materials and methods, were insulted by a sequence of tape-strippings. After 4 tape-strippings, TEWL from sites exposed to neutral pH buffer occurred at a rate of over 75 g/m²/h. In contrast, the sites exposed to an acidic, pH 5.5 buffer had TEWL rates close to 40 g/m²/h. The pattern persisted after five tape-strippings; here TEWL for the sites exposed to neutral buffer was about 90 g/m²/h and for site exposed to pH 5.5 buffer, TEWL was less than 75 g/m²/h.

Similarly, stratum corneum cohesion was reduced in skin treated with neutral pH buffer as compared to acidic pH buffer. Stratum corneum cohesion was measured as the cumulative protein removed with D-Squame stripping. After five sequential strippings, the amount of protein removed was about 30 μg/cm² for skin treated with neutral pH buffer and less that 25 μg/cm² for skin exposed to acidic pH buffer. These studies show that even short term exposure to neutral pH buffer produces functional abnormalities in stratum corneum integrity and cohesion.

### EXAMPLE 4

The experiments reported in this example demonstrate that exposure to an acidic pH buffer protects stratum corneum integrity from perturbation by inhibitors of secretory phospholipase A₂.

Murine skin that had been treated with the secretory phospholipase A₂ inhibitor BPB, for three days as described in materials and methods, was further exposed to either a neutral (pH 7.4) or acidic (pH 5.5) pH buffer for three hours. The increase in pH and abnormality in stratum corneum integrity provoked by the BPB treatment (see Example 1) was accentuated by exposure of the BPB treated site to neutral buffer for three hours. Exposure to the neutral buffer (pH 7.4) amplified the pH increase induced by BPB treatment from about pH 5.95 to about pH 6.41. In contrast, exposure to the acidic buffer (pH 5.5) for three hours overrode and reversed the inhibitor induced pH increase; the starting pH for the sites exposed to acidic buffer was about pH 5.95, and after exposure to the acidic buffer for three hours, the surface pH was reduced to about pH 5.8.

Stratum corneum barrier function as measured by rates of transepidermal water loss (TEWL), also revealed the beneficial effects of exposing the treated skin sites to an acidic pH buffer after treatment with BPB. Barrier function remained unchanged in BPB treated sites exposed for three hours to and acidic buffer, but skin sites exposed to a neutral buffer, showed an increase in the rate of TEWL from about 10 g/m²/h to about 18 g/m²/h.

Furthermore, stratum corneum integrity, as measured by the rate of TEWL induced by sequential tape stripping, was protected by exposure to the acidic buffer. After three sequential tape strippings of the BPB treated sites, the sites that were further exposed to acidic buffer experienced TEWL rates of less than 30 g/m²/h . In contrast, the treated and stripped sites that were exposed to neutral buffer, experienced TEWL rates of more than 60 g/m²/h.

The experiments reported in this example demonstrate that the abnormalities in stratum corneum pH, barrier function and integrity which are induced by secretory phospholipase A₂ inhibitor treatment, can be reversed or reduced by exposing the inhibitor treated sites to an acidic buffer. Thus, these results suggest that the abnormalities in stratum corneum integrity induced by secretory phospholipase A₂ inhibitor treatment can be attributed to the acidification abnormality produced by the inhibitor treatment.

### EXAMPLE 5

The experiments reported in this example demonstrate that the end products of secretory phospholipase A₂ hydrolysis of phospholipids, free fatty acids (FFA), are responsible for maintaining the acidic environment of the stratum corneum and thereby, protect its integrity and cohesion. Thus, application of the phospholipid substrate for secretory phospholipase A₂ hydrolysis or application of secretory phospholipase A₂ and a phospholipid substrate will protect the stratum corneum from the effects of increased pH.

Hairless mice were treated with BPB or MJ33 alone and in combination with free fatty acid for three days as described on materials and methods. Following treatment, surface pH of the skin was measured. Co-application to hairless murine skin of the secretory phospholipase A₂ inhibitor BPB and palmitic acid (PA), steric acid (SA) or to a lesser extent linoleic acid (LA), prevented the BPB induced increase in pH of the stratum corneum (PA: 5.40+/−0.14 vs. 5.92+/−0.05; SA: 5.73+/−0.09 vs. 6.17+/−0.08; LA: 5.77+/−0.10 vs. 6.28+/−0.1.). Furthermore, co-applications of BPB or MJ33 with palmitic acid, stearic acid and to a lesser extent linoleic acid also prevented emergence of secretory phospholipase A₂ inhibitor induced abnormalities in stratum corneum integrity and cohesion.

Mice were treated with BPB or MJ33 alone or in combination with free fatty acid for three days as noted above. Following treatment, the animals were tape stripped five times and the transepidermal water loss (TEWL) and protein removed per strip were measured for each of the five successive tape strips. As expected, treatment of Murine skin with BPB or MJ33 alone produced significant and progressive increases in TEWL rates in response to each tape strip, indicating impaired integrity. Co-application of one of the fatty acids along with BPB or MJ33 protected stratum corneum integrity; TEWL progressively increased with increasing numbers of tape strips, but the increase was significantly less than the increase seen for BPB alone. By the fifth strip, TEWL rates for sites treated with BPB alone were between 80-100 g/m²/h. Co-application of steric acid with BPB held the TEWL rates to about 25 g/m²/h. Co-application of palmitic acid and BPB kept the TEWL rates down to about 55-60 g/m²/h. Similarly, after three sequential tape-strippings, sites treated with MJ33 alone had TEWL rates close to 75 g/m²/h. Co-application of palmitic acid with MJ33 kept TEWL rates down to about 30-35 g/m²/h.

Co-application of fatty acids and one or the other secretory phospholipase A₂ inhibitor also protected stratum corneum cohesion. The cumulative protein removed by tape-stripping was significantly greater for the skin treated with BPB or MJ33 alone than it was for skin treated with BPB or MJ33 in combination with one of the fatty acids. By the fifth strip, sites treated with BPB alone lost an average of about 125 μg protein/cm². Co-application of stearic acid with BPB kept the cumulative protein loss down to about 25 μg/cm². Although somewhat less effective than steric acid, palmitic acid co-applied with BPB also reinforced the cohesion of the stratum corneum, keeping the cumulative protein loss at the fifth strip down to about 40 μg/cm². Similarly, sites treated with MJ33 alone lost about 50 μg protein/cm² after three sequential strippings whereas co-application of palmitic acid and MJ33 kept the loss of protein down to about 30 μg/cm² after three strippings.

These results show that the secretory phospholipase A₂-inhibitor-induced abnormalities in integrity and cohesion of the stratum corneum are linked to increased pH, and that the presence of the end products of phospholipid hydrolysis, fatty acids, protect the stratum corneum from the detrimental effects on integrity and cohesion that are associated with increased pH. Because free fatty acids are generated as an end product of phospholipid hydrolysis, application of the phospholipid substrate for secretory phospholipase A₂ hydrolysis or application of secretory phospholipase A₂ and a phospholipid substrate will protect the stratum corneum from the effects of increased pH.

### EXAMPLE 6

The experiments reported in this example demonstrate that consistent with its role in maintaining the neutral intracellular pH of keratinocytes and the acidic pH of the stratum corneum extracellular domains, the NHE1 antiporter is located in cultured human keratinocytes (CHK) and also in the differentiated cell layers of epidermis.

The human isoform of NHE1 was demonstrated to be present in both cultured human keratinocytes (CHK) and in epidermis by RT-PCR. As shown in materials and methods, primers were chosen so that amplification of a 505 base pair band would identify the human isoform of NHE 1. After isolation of mRNA from either epidermis or CHK as described in materials and methods, and RT-PCR, a cDNA product of the correct size (505 bp) and the correct sequence of that expected for the human isoform of NHE1 was generated.

NHE1 was also identified in preparations of CHK by western immunoblotting, as described in materials and methods, using an anti-NHE1 monoclonal antibody as a probe. The antibody identified a 114 kDa protein band, consistent with the predicted size of human NHE1.

Immunohistochemistry studies revealed NHE1 to be present in the outer nucleated layers of human epidermis, consistent with its proposed role in stratum corneum acidification. Human epidermal sections were stained with polyclonal NHE 1 antibody then detected with an FITC-labeled secondary antibody as described in materials and methods. Tissue was counterstained with propidium iodide then visualized by confocal microscopy. Immunolabeling could be localized to the cytosol of suprabasal cells in epidermis. The same staining pattern was observed in NHE1 wild type (+/+) mice, but not in NHE1 knockout (−/−) mouse epidermis.

The immunohistochemical localization studies, combined with the evidence obtained from RT-PCR and western-blotting, demonstrate that NHE1 is expressed in differentiated cell layers and keratinocytes, consistent with its role in maintenance of intracellular pH and acidification of stratum corneum extracellular domains.

### EXAMPLE 7

The experiments reported in this example demonstrate that the NHE1 antiporter is an important factor maintaining the intracellular pH in cultured human keratinocytes (CHK) and that NHE1 also contributes significantly to maintenance of an acid pH environment of the stratum corneum. The role of NHE1 in pH regulation in both CHK and the stratum corneum was investigated using two inhibitors specific for the NHE1 sodium proton exchanger, amiloride and HOE694.

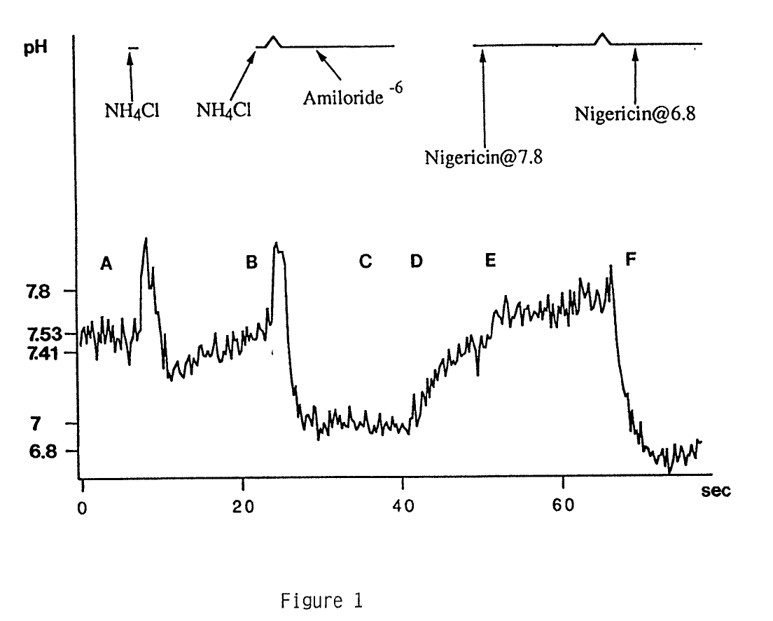

Activity of the NHE1 antiporter was tested by measuring the cells ability to recover from an acid load. As can be seen in FIG. 1, when NH₄Cl is added to cultures of CHK, the cells initially become more basic and, after dissociation of the ammonium ion, take on an acid load. Cells slowly extrude H⁺ re-establishing a more neutral pH. The addition of the specific NHE1 inhibitor, amiloride, to the culture at 1 μM, blocks the recovery of the cells from the acid load (FIG. 1). Consistent with this observation, when the NHE1 inhibitor HOE694 was applied at a concentration of 1.5 μM for 16 hours, the intracellular pH dropped from pH 7.05 to 6.90, suggesting that the cells are not able to pump out the extra H⁺ ions to normalize the intracellular pH. These results show that NHE1 is present in human keratinocytes, and that NHE 1 activity regulates intracellular pH.

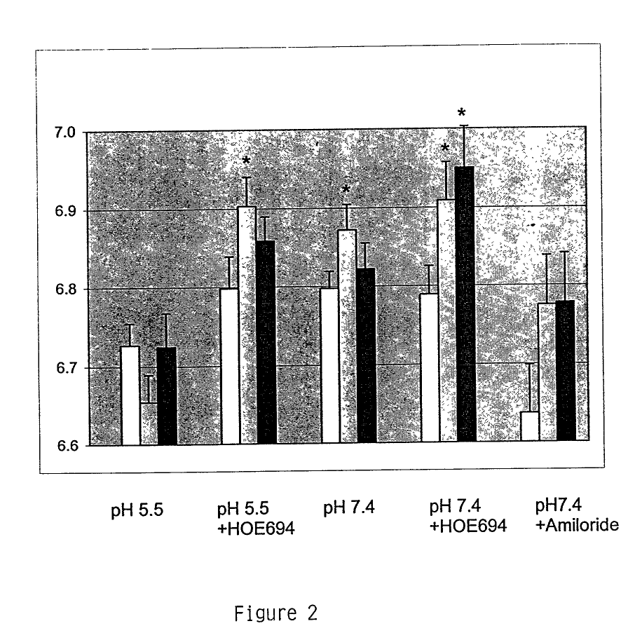

To investigate the role of NHE 1 in maintaining the pH balance of the stratum corneum, the integrity of murine skin exposed to different buffers and NHE1 inhibitors, was disrupted by tape-stripping. The pH of the exposed skin was measured before and after tape-stripping. Before tape-stripping, the surface pH in all animals was acidic (6.03+/−0.20, n=78). Tape stripping invariably resulted in an alkalinization of stratum corneum (6.77+/−0.15, n=156). Relative to the initial post-tape-strip time point, skin exposed to a HEPES based pH 5.5 buffer, became more acidic at 2 hrs post-tape-strip. At 5 hours post-tape strip the skin exposed to pH 5.5 buffer returned to its initial post tape strip value, suggesting that the stratum corneum had recovered from the initial acid load (FIG. 2). When the NHE1 inhibitor, HOE694, was added to the pH 5.5 buffer, the initial, post-tape-strip pH value was increased significantly with respect to the initial post-tape-strip value of skin exposed to pH 5.5 buffer alone. This result suggests that the mechanism responsible for acidifying the stratum corneum is inhibited. At two hours post-tape-strip, the skin exposed to HOE694 in pH 5.5 buffer was more alkaline than the initial post-tape-strip value by more than 0.1 pH units, strongly suggesting that the mechanism that acidifies the stratum corneum is blocked. At five hours post-tape-strip, these samples were still not fully recovered, remaining more than 0.05 pH units higher than the initial post-tape-strip values. Thus, these data suggest that the NHE1 antiporter has a significant effect in maintaining the acid environment of the stratum corneum.

Consistent with the results of the above described experiment, exposure of murine skin to pH 7.4 buffer resulted in a pH value at two hours post-tape strip that was increased by 0.075 pH units over the initial post-tape strip value. However, at five hours post tape strip, the pH of the skin exposed to pH 7.4 buffer was nearly recovered to its initial post-tape-strip value, suggesting that the NHE1 antiporter is still functioning. When the NHE1 inhibitor HOE694 was added to the pH 7.4 buffer, the initial post-tape-strip pH value was similar to the pH of the skin exposed to buffer only. However, at two hours post tape strip, the pH had increased significantly, being more that 0.1 pH unit higher than the initial post-tape-strip value and close to 0.05 pH units higher than the two hour time point of skin exposed to pH 7.4 buffer alone. Unlike the case for skin exposed to pH 7.4 buffer only, this elevated pH persisted and even increased at five hours post tape strip, suggesting that the mechanism that acidifies the stratum corneum is strongly blocked (FIG. 2).

These experiments demonstrate that the activity of the NHE1 antiporter is required in order to maintain the acidic pH of the normal stratum corneum. They also demonstrate that the activity of the NHE1 antiporter is stimulated by the application of an acid load, in the form of NH₄⁺ ions. As the NH₄⁺ ion enters the cells of the outer epidermis, it is reduced to NH₃, which evaporates. The excess H⁺ lowers the intracellular pH, activating the NHE1 antiporter to expel the excess H⁺. Expulsion of excess H⁺, in turn, leads to extracellular acidification of the outer epidermis and stratum corneum. Thus, NHE1 couples cellular physiology and the extracellular environment.

### EXAMPLE 8

The experiments reported in this example demonstrate that NHE1 mediated acidification is linked to function of the stratum corneum.

Transepidermal water loss (TEWL) was measured in order to assess the kinetics of barrier recovery following acute barrier perturbations induced by tape-stripping. Both of the NHE1 inhibitors, amiloride and HOE694, delayed barrier recovery.

Hairless mice were tape stripped to TEWL of 7-9 g/m²/h. Hilltop Chambers with or without amiloride at 5 μM or HOE694 at 7.5 μM in 10 mM HEPES buffer adjusted to pH 7.4 or pH 5.5 buffer were applied. Control areas were covered with Hilltop Chambers containing 10 mM HEPES buffer adjusted to pH 7.4 or pH 5.5 as appropriate. TEWL was measured at 0, 2, 5, and 24 hours post tape-strip. The results are reported as percent barrier recovery from the initial induced defect.

When either amiloride or HOE694 were applied in pH 7.4 buffer, barrier recovery was delayed relative to recovery of the skin treated with pH 7.4 buffer alone. At two hours post tape-strip, the skin treated with pH 7.4 buffer alone experienced a TEWL rate 21% less than the initial induced TEWL rate. In contrast, skin treated with HOE 694 or amiloride in pH 7.4 buffer had recovered to TEWL rates only about 12% less than the initial induced TEWL rate. Similarly, at five hours post tape-strip, the skin treated with pH 7.4 buffer had recovered to a TEWL rate about 38% less than the initial induced TEWL rate, while the skin treated with HOE 694 and pH 7.4 buffer had only recovered to a TEWL rate 23% less than the initial induced TEWL rate and the skin treated with amiloride and pH 7.4 buffer had a TEWL rate only about 25% less than the initial rate induced by tape-stripping. The delay in barrier recovery was especially pronounced at 24 hours post tape-strip, at which point the skin treated with pH 7.4 buffer only, recovered to TEWL rates 75% less than the initial TEWL rate induced by tape-stripping. In contrast, skin treated with HOE 694 in pH 7.4 buffer recovered to TEWL rates only about 45% less than the initial induced TEWL rate.

Unlike the case of skin treated with HOE 694 in pH 7.4 buffer, skin treated with HOE694 in pH 5.5 buffer, recovered as well as the skin sites treated with pH 5.5 buffer only. A slight delay in barrier recovery was apparent at 2 hours post tape-strip, where skin sites treated with HOE 694 in pH 5.5 buffer had recovered to TEWL rates 25% less than initial rate induced by tape-stripping, whereas skin sites treated with pH 5.5 buffer alone recovered to TEWL rates 28% less than the initial induced rate. After the two hour time point, skin sites treated with HOE 694 in pH 5.5 buffer, as well as sites treated with pH 5.5 buffer and sites treated with pH 7.4 buffer recovered in parallel, experiencing TEWL rates 38% less than the initial induced value by 5 hours post tape-strip, and TEWL rates 75% less than the initial induced TEWL rate at 24 hours post tape-strip.

Thus, the pH of the applied buffer is less critical for barrier recovery when the NHE1 antiporter is functional. However, treatment of skin sites with pH 5.5 buffer facilitates barrier recovery in the presence of the NHE1 inhibitor. This suggests that the delay in barrier recovery seen in the skin treated with HOE 694 in pH 7.4 buffer is due to an alteration in stratum corneum pH which, in the absence of NHE1 function, can be manipulated by application of buffers of different pH values.

### EXAMPLE 9

The experiments reported in this example demonstrate that permeability barrier homeostasis is abnormal in transgenic NHE1 knockout mice.

NHE1 knockout mice were generated via gene targeting to eliminate NHE1. Barrier function was first assessed under basal conditions and neither the surface pH nor the baseline transepidermal water loss (TEWL) rates differed significantly between NHE −/− and +/+ littermates. However, after the integrity of the epidermal barrier was challenged by tape-stripping, differences in barrier recovery became apparent. Barrier recovery was significantly delayed in −/− mice compared with their wild type littermates, especially at 5 and 8 hours post tape-strip.

Paired mice were shaved and tape-stripped as in Example 8 above. Barrier recovery was then measured as percent recovery from the intial defect. At five hours post tape strip, barrier function in wild type (+/+) mice had recovered to TEWL rates that were 30% less than the initial defect, whereas their −/− littermates recovered to TEWL rates that were only 20% less than the initial TEWL rates induced by tape-stripping. Similarly, at 8 hours post tape-strip, wild type mice had recovered to TEWL rates that were 45% less than the TEWL rate induced by tape-stripping, whereas their −/− littermates recovered to TEWL rates that were only 30% less than the initial defect. The pattern of recovery resembled that seen with the topical inhibitor HOE694 in pH 7.4 buffer described in Example 8 above.

Thus, these results demonstrate that the NHE1 antiporter is important for normal recovery of barrier function following disruption by tape-stripping and that NHE1 is important for facilitating this recovery.

### EXAMPLE 10

The experiments reported in this example demonstrate that the inhibition of NHE1 activity impedes barrier recovery through its effect on extracellular processing of secreted stratum corneum lipids.

The mechanistic basis for the inhibitor and NHE 1 knockout induced delays in barrier recovery (described in Examples 8 and 9, respectively) was investigated using electron microscopy. Two competing hypotheses were tested. The delay in barrier recovery could be due to defects in lamellar body formation and secretion of stratum corneum lipids or instead, could be due to extracellular processing of stratum corneum lipids following secretion.

Biopsies were taken during time course experiments. When EM images from animals treated with HOE694 in a pH 7.4 buffer were assessed, it was found that lamellar body formation and secretion were undisturbed. However, the persistence of incomplete, immature extracellular lamellar bilayer structures suggested that extracellular processing of secreted lipids was defective. Interestingly, when biopsies of animals treated with HOE694 in pH 5.5 buffer were viewed under the electron microscope, extracellular processing appeared normal. These results suggest that inhibition of NHE1 activity impedes barrier recovery by interfering with extracellular processing of secreted stratum corneum lipids, and that this effect can be overridden by the application of an acidic (pH 5.5) buffer.

Similarly, electron micrographs of biopsies taken from NHE1 −/− knockout mice revealed the persistence of newly secreted lipids several layers above the stratum corneum-stratum granulosum interface, and the presence of incompletely processed lamellar membrane structures. Thus, extracellular lipid processing appears to be delayed in NHE1 knockout (−/−) mice in the same way that it is in mice that have been treated with the NHE1 inhibitor, HOE694, in neutral buffer.

These results show that the skin of NHE1 knockout mice and the skin of mice treated with the NHE1 inhibitor, HOE694, in pH 7.4 buffer both have similar defects in extracellular processing of secreted stratum corneum lipids. Interestingly, in the case of inhibitor treated animals, this defect in extracellular processing of secreted lipids is reversed by the application of an acidic buffer.

The foregoing is offered primarily for purposes of illustration. It will be readily apparent to those skilled in the art that the concentrations, conditions of administration, and other parameters of the invention as described herein may be further modified or substituted in various ways without departing from the spirit and scope of the invention.

What is claimed is:

1. A method for treating the epidermis of a terrestrial mammalian subject suffering from a perturbed epidermal barrier function, said method comprising administering to said epidermis a topical composition comprising an active ingredient that acidifies the pH of the stratum corneum and is a member selected from the group consisting of phospholipases, phospholipids, salts with monovalent cations, and buffers with a pH less than 7.0, said active ingredient being present in a concentration that is effective in acidifying the pH of said epidermis and thereby improving barrier function.

2. A method in accordance with claim 1 in which said active ingredient is a phospholipase.

3. A method in accordance with claim 2 in which said phospholipase is a member selected from the group consisting of phospholipase A₁ and phospholipase A₂.

4. A method in accordance with claim 2 in which said phospholipase is a natural or bioengineered 14 kDa type 1 secretory phospholipase A₂.

5. A method in accordance with claim 2 in which said enzyme is type 1 secretory phospholipase A₂ pancreatic type.

6. A method in accordance with claim 1 in which said active ingredient is a phospholipid.

7. A method in accordance with claim 6 in which said phospholipid is a non-essential fatty acid containing phospholipid.

8. A method in accordance with claim 6 in which said phospholipid is dipalmitoylphosphatidylcholine.

9. A method in accordance with claim 1 in which said active ingredient is a salt with a monovalent cation and with the proviso that the counter ion is not lactate.

10. A method in accordance with claim 9 in which said salt is an ammonium salt.

11. A method in accordance with claim 9 in which said salt is a member selected from the group consisting of ammonium chloride, ammonium phosphate, ammonium carbonate, ammonium nitrate, ammonium sulfate, ammonium sulfonate, ammonium fluoride, ammonium iodide and ammonium bromide.

12. A method in accordance with claim 9 in which said salt is ammonium chloride.

13. A method in accordance with claim 9 in which said salt is a member selected from the group comprised of ammonium citrate, ammonium tartrate and ammonium acetate.

14. A method in accordance with claim 9 in which said salt is a sodium salt.

15. A method in accordance with claim 1 in which said active ingredient is a buffer solution with a pH less than 7.0.

16. A method in accordance with claim 15 in which said buffer solution is a member selected from the group consisting of solutions of HEPES-based, MES-based, MOPS-based, PIPES-based, TES-based, phosphate-based, citrate-based and bicarbonate-based buffers.

17. A method in accordance with claim 15 in which said buffer solution is a solution of a HEPES-based buffer.

18. A method in accordance with claim 15 in which said buffer solution is in the pH range of 4.5-6.5.

19. A method in accordance with claim 15 in which said buffer solution is a pH 5.5 buffer.

20. A method in accordance with claim 1 in which said topical composition comprises two or more of said active ingredients.

21. A method for treating the epidermis of a terrestrial mammalian subject suffering from an impaired epidermal cohesion, said method comprising administering to said epidermis a topical composition comprising an active ingredient that acidifies the pH of the stratum corneum and is a member from the group consisting of phospholipases, phospholipids, salts with monovalent cations, and buffers with a pH less than 7.0, said active ingredient being present in a concentration that is effective in acidifying the pH of said epidermis and thereby improving epidermal cohesion.

22. A method in accordance with claim 21 in which said active ingredient is a phospholipase.

23. A method in accordance with claim 22 in which said phospholipase is a member selected from the group consisting of phospholipase A₁ and phospholipase A₂.

24. A method in accordance with claim 22 in which said phospholipase is a natural or bioengineered 14 kDa type 1 secretory phospholipase A₂.

25. A method in accordance with claim 22 in which said phospholipase is type 1 secretory phospholipase A₂ pancreatic type.

26. A method in accordance with claim 21 in which said active ingredient is a phospholipid.

27. A method in accordance with claim 26 in which said phospholipid is a non-essential fatty acid containing phospholipid.

28. A method in accordance with claim 26 in which said phospholipid is dipalmitoylphosphatidylcholine.

29. A method in accordance with claim 21 in which said active ingredient is a salt with a monovalent cation and with the proviso that the counter ion is not lactate.

30. A method in accordance with claim 29 in which said salt is an ammonium salt.

31. A method in accordance with claim 29 in which said salt is a member selected from the group consisting of ammonium chloride, ammonium phosphate, ammonium carbonate, ammonium nitrate, ammonium sulfate, ammonium sulfonate, ammonium fluoride, ammonium iodide and ammonium bromide.

32. A method in accordance with claim 29 in which said salt is ammonium chloride.

33. A method in accordance with claim 29 in which said salt is a member selected from the group comprised of ammonium citrate, ammonium tartrate and ammonium acetate.

34. A method in accordance with claim 29 in which said salt is a sodium salt.

35. A method in accordance with claim 21 in which said active ingredient is a buffer with a pH less than 7.0.

36. A method in accordance with claim 35 in which said buffer solution is a solution of a member selected from the group consisting of HEPES-based, MES-based, MOPS-based, PIPES-based, TES-based, phosphate-based, citrate-based and bicarbonate-based buffers. buffers.

37. A method in accordance with claim 35 in which said buffer solution is a solution of a HEPES-based buffer.

38. A method in accordance with claim 35 in which said buffer solution is in the pH range of 4.5-6.5.

39. A method in accordance with claim 35 in which said buffer solution is a pH 5.5 buffer.

40. A method in accordance with claim 21 in which said topical combination comprises two or more of said active ingredients.
